(19) United States
(12) Patent Application Publication
(10) Pub. No.: US 2002/0090606 A1
(43) Pub. Date: Jul. 11, 2002

(54) Method and specific phage library for high throughput proteomics

(76) Inventors: Sandy Stewart, Durham, NC (US); John M. Crawford JR., Raleigh, NC (US)

Correspondence Address: PARADIGM GENETICS, INC, 104 ALEXANDER DRIVE, BUILDING 2, P O BOX 14528, RTP, NC, 27709-4528, US

(21) Appl. No.: 09/757,050

(22) Filed: Jan. 8, 2001

(51) Int. Cl. C12Q 1/70; C12N 7/00

(52) U.S. Cl. 435/5; 435/235.1

(57) ABSTRACT

Immunocompentent phage display libraries, specific B-cells, lymphocytes, antibodies and methods for their preparation, useful for high throughput screening and other useful applications in proteomics are described.

### FIELD OF THE INVENTION

The present invention relates to immunocompetent phage display libraries, specific B-cell lymphocytes, antibodies and methods for their preparation thereof, useful for high throughput screening and other useful applications.

### BACKGROUND OF THE INVENTION

It is widely accepted that the expression of mRNA, even at high levels, does not necessarily correspond to translation into protein. Conventional phage display antibody (Ab) techniques utilize commercially available mouse naïve antibody (Ab) genes for production. One of the greatest disadvantages to using phage libraries is that the antibodies recovered require dozens of rounds of intensive screenings and amplifications and result in antibodies with a notoriously low binding affinity. This low binding affinity is due to the fact that the libraries are considered “naïve” since no form of pre-selection against antigens of interest have occurred and thus no affinity maturation has occurred. Thus, a method was sought that would reduce the rounds of intensive screenings and amplifications in order to provide a more high-throughput process for preparing an immunocompentent (i.e. non-naive) phase display libraries that would advance the field of proteomics.

### SUMMARY OF THE INVENTION

In one embodiment, the present invention is directed towards a method for preparing an immunocompetent phage display library comprising:

a) injecting into an animal,a source of antigen(s) from an organism of interest;

b) allowing said animal to develop specific B-cells expressing predominately (primarily) immunoglobin IgG antibody against said antigen(s);

c) withdrawing a blood sample from said animal containing said B-cells expressing immunoglobin on their cell surface;

d) immobilizing the source of antigen(s) on a support or solid surface;

e) contacting said blood or blood component with said support or solid surface to capture said specific B-cells expressing IgG directed against said antigen(s) of interest from said blood or blood component; and

f) recovering said specific B-cells from said support or solid surface; and

g) preparing an immunocompetent phage display library from said recovered specific Bcells.

In another embodiment, the present invention is directed towards a method for preparing an immunocompetent or non-naive phage display library, comprising:

a) mounting an immune response in an animal, with an a source of multiple antigens from a tissue of interest to generate specific B-cells that have undergone in vivo somatic hypermutation,

b) recovering specific B-cells from the blood or blood component of the immunized animal, and

c) preparing an immunocompetent phage display library that displays antibody proteins or fragments thereof, from the recovered, specific B-cells.

In another embodiment, the present invention is directed towards a immunocompetent phage display libraryper se prepared by the methods described above and herein.

In another embodiment, the present invention is directed toward a method for screening for phages within an immunocompetent phage display library comprising:

a) mounting an immune response in an animal with an a source of antigen(s) from an organism of interest to generate specific B-cells that have undergone in vivo somatic hypermutation,

b) isolating said specific B-cells from the blood or blood component of the immunized animal,

c) preparing a phage display library that displays antibody proteins or fragments thereof from said specific B-cells,

d) separating said source of antigen(s) into individual proteins or peptides thereof, and

e) screening or selecting for phages within said immunocompetent phage display library that bind with said separated antigen proteins or peptides.

In another embodiment, the present invention directed toward a method for characterizing either the phages within an immunocompetent phage display library and/or its antigen source, comprising:

a) mounting an immune response in an animal with an a source of antigen(s) from an organism of interest to generate specific B-cells that have undergone in vivo somatic hypermutation;

b) recovering said specific B-cells from the blood or blood component of the immunized animal;

c) preparing a phage display library that displays antibody proteins or fragments thereof, from said specific B-cells,

d) separating said source of antigen(s) into individual proteins or peptides thereof,

e) screening or selecting for phages within said immunocompetent phage display library that bind with said separated antigen proteins or peptides, and

f) characterizing:

i) the protein sequence and/or molecular weight of the separated antigen proteins binding to the phage and/or

ii) the nucleic acid sequence within the binding phage that codes for the antibody protein or fragment thereof.

In another embodiment, the present invention is directed toward a method for preparing a library of antibodies and/or fragments thereof, comprising:

a) injecting into an animal a source of antigen(s) from an organism of interest;

b) allowing said animal to develop specific B-cells expressing predominately immunoglobin IgG antibodies;

c) withdrawing a blood sample from said animal containing said specific B-cells;

d) immobilizing the source of antigen(s) to a support or solid surface;

e) contacting said blood sample or component thereof with said support or solid surface to remove said specific B-cells from said blood or blood component;

f) isolating RNA from said specific B-cells;

g) reverse transcribing said RNA to produce cDNA;

h) inserting said cDNA into a phage display vector that expresses antibodies on the phage surface,

i) isolating phage which bind to said source of antigen(s), and

j) obtaining a library of antibodies or fragments thereof from said phage.

In another embodiment, the present invention is directed toward a method for preparing a non-naive or immunocompetent library of antibodies and/or fragments thereof, comprising:

a) mounting an immune response in an animal with an a source of antigen(s) from a tissue of interest to generate specific B-cells that have undergone in vivo somatic hypermutation,

b) isolating specific B-cells from the blood or blood component of the immunized animal,

c) preparing an immunocompetent phage display library that displays antibody proteins or fragments thereof from said specific B-cells,

d) separating said source of antigen(s) into individual proteins or peptides thereof,

e) screening or selecting for phages within said immunocompetent phage display library that bind with said separated antigen proteins or peptides, and

f) obtaining antibodies or fragments thereof from said binding phage.

In another embodiment, the present invention is directed toward the antibodies per se prepared by the methods described above and herein.

Thus, the present invention provides a method for preparing antibody based proteomics chips specific for various cells, tissues, tumors, diseases, organisms, organs, etc., which can be used in a wide variety of health, pharmaceutical, microbial, diagnostics, agricultural and nutritional applications. For example, in the field of drug discovery the present invention can be used to identify potential targets through protein differences between normal and diseased states. Similarly, the present invention can also be used to deduce the mode of action of a test compounds for novel and known targets. The present invention can also be used to increase the efficiency and predictive power of lead efficacy and toxicological testing. For example, changes in biochemical pathways of an organism can be determined by comparing protein expression of subjects receiving treatment with a specific drug versus untreated subjects. The present invention can also be used to deduce microbial protein profiling associated with drug resistance, such as has been demonstrated with S. pneum to erthromycin in which glyceraldehyde-3-phosphate dehydrogenase is significantly altered. The present invention can also be used a diagnostic tool to diagnose diseases, disease predisposition, drug interactions and individual interaction/indications. Further, the immunocompetent antibody library of the present invention can be utilized for drug targeting to specific tumors, tissues, and as imaging markers in addition to the possibility of humanized antibody therapeutics which important therapeutic uses. The immunocompetent antibodies of such libraries can also assist in drug design due their specificity in epitope binding.

Additionally the antibody library can be used for a host of additional studies including mode of action, receptor binding, and assay development. The antibodies in such library would be useful for diagnostics and humanized Ab therapy since the binding domain gene sequence could be cloned into humanized mice Ab genes. Thus, the present invention is completely amenable to any organism or tissue that one could investigate.

### DETAILED DESCRIPTION OF THE INVENTION

“Antibody” refers to an immunoglobulin protein whether naturally or synthetically produced which is capable of binding an antigen. When they are naturally produced, antibodies are secreted into the bloodstream to seek out and bind foreign agents or antigens for destruction. The term also covers any protein having a binding domain that is homologous to an immunoglobin binding domain.

“Antibody fragment thereof” or “antibody protein fragment thereof” refers to that portion of an antibody (i.e. Fv) capable of binding to an antigen.

“Antigen” refers to a foreign substance that, upon introduction or injection into a vertebrate animal such as a mammal or poultry, stimulates the animal to produce antibodies as a defensive measure that can combine or bind with the antigen. Such antigens can be derived from a broad range of sources and can include, for example, viruses, proteins, nucleic acids, organic compounds and the like.

“Antigen beads” refers to magnetic beads that have been covalently linked to antigens from a particular tissue or tissue extract. Typically the peptide antigens are conjugated to the beads at the carboxyl (C-terminus) and the amino (N-terminus) using a suitable conjugating agent, such as EDC or SMCC.

“Antibody gene” refers to a section of DNA that codes for an antibody protein or fragment thereof.

“Affinity” refers to the strength of binding between one antibody (Ab) molecule and its respective antigen. A strong binding affinity between the Ab and its respective antigen is desired. A K constant, determined experimentally, can be used to measure or evaluate affinity.

“B-cells”, also known as “B lymphocytes” refers to spherical white blood cells that produce antibodies which are responsible for most immunological reactions. A single B-cell produces only one antibody (Ab) that recognizes only one single epitope. Specific B-cells are B-cells that have undergone in vivo somatic hypermutation and express predominately immunoglobin IgG antibody.

“DNA” refers to deoxyribonucleic acid.

“Epitope” refers to that part of an antigen or antigenic molecule against which a particular immune response is directed. The terms “epitope” and “antigenic determinant” may be used interchangeably. In the animal, most antigens will present several or even many epitopes or antigenic determinants simultaneously, depending on the size and immunogenicity of the antigen or antigenic molecule.

“Immunoaffinity purification” such as affinity chromatography” is a technique that can be used to select an antibody that binds to an antigen. In this technique, the antibodies are used either as the immobilized or mobilized molecules to capture its antigen. For example, phage antibodies can be covalently linked or immobilized to a support or column, sometimes known as an immunoaffinity column, where antigens can be separated from a complex mixture. Conversely, antigens can be immobilized to a support or column, where phage antibodies can be separated from a complex mixture.

“Immunocompetent” refers to a phage display library prepared from specific or non-naive B-cells.

“Monoclonal antibodies” are immunoglobins derived from clones of a single B-cell. Monoclonal antibodies are produced by a single clone of hybridomas cells, and are therefore a single species of antibody molecule. Because the antibody-secreting hybridoma cell line is immortal, due to in vitro fusion of a B-cell with a tumor cell, the characteristics of the antibody are reproducible from batch to batch. The key proprieties of monoclonal antibodies are their specificity for a particular antigen and the reproducibility with which they can be manufactured.

“mRNA” refers to messenger RNA.

“Naive animal” refers to an animal that has not been previously immunized by a specific antigen.

“Naive antibodies” refers to antibodies produced in vitro within a phage display system.

“Non-naive animal” refers to an animal that has been previously immunized with a source of antigens. Suitable animals for immunizing the source of antigens with include, but are not limited to mammals such as humans, sheep, cattle, rabbits, goats, dogs, cats, mice and rats; and to birds or poultry such as chickens, geese and turkeys.

“Non-naive phage display library” refers to an immunocompetent phage display library prepared from B-cells which have been previously exposed to a source of antigens and have undergone in vivo somatic hypermutation.

“Nucleic acid sequence” refers to the nucleotide sequence of DNA or RNA.

“Organism” refers to a life form from any of the kingdoms, such as animalia, planta, protista, monera, fungi, and archae that can provide a source of antigen(s) through itself or any part or tissue thereof. Such tissues can include but are not limited to cells, organs or fluids, such as blood or spinal fluid.

“Panning” refers to a technique used to select an antibody that binds to an antigen from a phage display library. Typically, one of the partner antigen molecules is immobilized to a solid surface or coupled to a carrier protein prior to coating the solid surface. A suspension containing the library of mobile phage antibodies is swirled over the surface like a gold-rush panner looking for gold in river silt. If any of the mobile antibody molecules bind to the surface, a match has been found. The unbound phage antibodies are removed by washing.

“PCR” refers to polymerase chain reaction, a technique for copying the complementary strands of a target DNA molecule simultaneously for a series of cycles until the desired amount is obtained. First, primers are synthesized that have nucleotide sequences complementary to the DNA that flanks the target region. The DNA is heated to separate the complementary strands and then cooled to let the primers bind to the flanking sequences. A heat-stable polymerase is added, and the reaction is allowed to proceed for a series of replication cycles.

“Phage” refers to a bacteriophage or virus that infects bacteria. Basically, a phage consists of a protein coat or capsid enclosing the phage genome or genetic material (DNA or RNA) which is injected into the bacterium upon infection. This injected genetic material then directs the bacteria to synthesize the phage's genetic material and proteins using the host bacteria's transcriptional and translational apparatus. These phage components then self-assemble to form new phage viruses or particles.

“Phage display” is a method of using phages to make and test proteins, particularly newly generated proteins such as antibodies or fragments thereof. A gene encoding a protein is cloned into a phage genome or genetic material in such a way that the protein appears on the surface of the phage. The phage can be selected by selecting the protein directly using panning or affinity chromatography. The protein that is selected is still bound to the phage so that the phage can be grown to identify the gene sequence, and hence the protein sequence, to manipulate it further.

“Phage display library” refers to a collection of nucleic acids that have been inserted into phage vector and that code for antibody genes or portions thereof. In the present invention, the genes are derived from B-lymphocytes produced by non-naive animals in response to immunization from a source of multiple, non-purified antigens. The library can contain a few or a large number of different nucleic acid molecules, varying from about ten molecules to several billion molecules or more. If desired, a molecule or a phage vector can be linked to a tag, which can facilitate recovery or identification of the molecule.

“Proteomics” refers to the scientific discipline that attempts to catalog and characterize proteins and polypeptides, compare variations in their expression levels under different conditions (i.e. sickness versus health), study their interactions and identify their functional roles.

“RNA” refers to ribonucleic acid.

“Phage vector” is a bacterial virus which can receive the insertion of a gene or other genetic material, resulting in a recombinant DNA molecule. The phage vector is capable of self-replication in a host organism. A phage vector contains an origin of replication for a bacteriophage but not for a plasmid.

“Serum” or (sera, plural) refers to the fluid remaining after blood clots or coagulates.

“Support” refers to a defined surface to which an antigen or antibody can be covalently linked or immobilized, either directly or indirectly.

Generalized Procedure and Theory

The present invention uses phage display libraries that are considered “non-naive” or immunocompetent where natural, in vivo somatic hypermutation has occurred whereby an antibody producing B-cell undergoes genetic rearrangement, generally resulting in an isotype change and a stronger antigen-antibody binding affinity than found in “naive” phage display libraries. The present invention utilizes antibodies for the detection and identification of proteins or peptides in a truly high throughput mechanism, for example, by utilizing a immunocompetent phage display antibody library micro-arrayed in a chip format. A novel method is used to generate the genetic material required for phage infection. The method of the present invention involves mounting the immune response in an animal with a source of antigen(s) to generate B-cells that undergo in vivo somatic hypermutation. After a sufficient titer has developed, blood is withdrawn from the animal and total B-cells are isolated from the whole blood. Larger animals tend to be more useful for drawing whole blood to isolate enough B-cells for this process, generally about 50 ml or more of whole blood or blood component. These B-cells are then incubated with magnetic beads that have been covalently coated with the antigen(s) of interest and therefore only the B-cells expressing Ab (on their cell surface) against antigen(s) (Ag) of interest will be isolated. The DNA from these immunocompetent, class-switched, high affinity B-cells will be used to generate the phage display library. This will result in a specific, immunocompetent library, eliminating the need for multiple rounds of panning and screening procedures, whereby the Ab will be predominantly high affinity and extremely useful. Antibodies such as these can be used more multiple purposes, such as, but not limited to proteomics, as described above, mode of action studies, diagnostics, imaging and assay development.

The library can be screened against 2-D gels or proteins excised from 2-D gels of the tissue of interest for characterization and/or amplification or the library for differential studies (see below). That is, the phage display library is screened or selected for phages within the library that bind to the separated antigen proteins. The screening itself can be done via the tissue extracts (i.e. the source of multiple antigens) run on a 2-D gel to separate the multiple antigens into individual protein or peptide components. The protein or peptide components are transferred to a membrane such as PVDF, stained with a staining agent such as Ponceau-S to visualize the spots, and then hybridized with the entire library of phage. The Ponceau-S spots can be excised and DNA, if present, is eluted off to obtain the phage producing the antibody of interest. This results in a highly useful library of characterized Abs that can be utilized in a variety of techniques including proteomics on a chip (protein expression profiling-PEP). Also, once the animal Ab genes have been isolated and cloned and the subsequent techniques are perfected, this technology can be repeated for any tissue of interest quite rapidly.

Differential studies can be accomplished by using a library from a normal or wild-type individual and comparing this to mutants or abnormal individuals. In such a case one would not be required to know which protein that each antibody recognizes. The antibodies could be arrayed in a defined template such that any difference/variance between samples would be identified visually. The variant protein is then isolated using the antibody against it and it would then moved ahead for characterization of the protein as defined above.

The protein sequence and/or molecular weight can be determined or characterized for the separated antigen proteins that bind to the phage. Also, the nucleic acid sequence (i.e. cDNA) within the binding phage that codes for the antibody or fragment thereof can also be determined or characterized.

Theory

While applicant(s) do not wish to be bound to any specific theory to explain why the present invention is effective in preparing non-naive phage display libraries and chips, the following theoretical explanation can be given.

In vivo the first introduction of antigen to an antibody producing cell (B-cell) by an antigen presenting cell (APC), such as a macrophage or T-helper cell (Th-cell) results in antibody being expressed that is typically of the IgM class and has a low affinity for this specific antigen, e.g., its binding strength is low. However, a second and multiple introductions result in a process known as somatic hypermutation (class-switching) whereby an actual genetic rearrangement occurs in the B-cell for production of a different class of Ab, predominantly IgG, that typically has a much higher binding affinity as well due to the genetic rearrangement. Unfortunately, this key step is extremely difficult to be duplicated in vitro. In vitro immunization of B-cells only results in the first stage of the process whereby the initial presentation occurs.

Our intention is to generate a phage display Ab library that is immunocompetent, not naive, for a tissue of interest and that has experienced class-switching/post-somatic hypermutation. Such a library is not available commercially.

Initiating the Immune Response

The immune system involves producing different classes of antibodies (Abs), each class defined by a different heavy chain. IgM and IgG, which carry the mu and gamma heavy chains, respectively, are secreted into the bloodstream , where they recognize circulating antigens. Complexes of antigen with IgM or IgG activate the complement system, a set of proteins that kills cells to which the antibody is bound. These complexes also activate cells called macrophages that actively engulf and destroy bacteria and other antigens. Immunocompetent or immune phage display libraries can be created because of a phenomena known as class switching, i.e. the maturation of an immune response in which, for example, B-cells undergo genetic rearrangement known as post-somatic hypermutation whereby, for example, the genes encoding the heavy chains can be rearranged to to begin encoding for IgG isotypes instead of IgM isotypes. In addition affinity maturation is thought to occur through the internalization of foreign proteins bound to immunoglobulin receptors on the B-cell surface which are then digested into peptides and associated with invariant chain and Ig complexes resulting in genetic rearrangement of genes coding for the epitope binding region of the Ig. This rearrangement occurs to produce a higher binding affinity Ig against the most immunogenic region of the foreign peptide. The present invention provides a method which mounts an immune response in an animal to generate antibody producing B-cells. The antigen of interest is prepared or formulated to serve as a source for immunization for the animal species of interest. The antigen can be derived from virtually any source, including but not limited to, plant, microorganism, animal, chemical, spore, or organic molecules. The antigen can also be in various states of purity or multiple complex mixtures of non-purified antigens. The source of multiple antigens is not limited to a few antigens as for a purified antigen, but rather, can contain tens, hundreds or even thousands of antigens from the source of interest. Typically, the animal species chosen for immunization is injected with the antigen in a mixture of adjuvant following a specific protocol. Adjuvant is an immune response booster and is typically composed of a mixture of mineral oil and killed mycobacterium. One of the most widely used adjuvants of this type is called Freund's and is typically found as both “Complete” and “Incomplete.” The injection protocol is designed to eventually elicit B-cells that produce predominantly high affinity IgG immunoglobins.

After immunization, the reactivity of the animal's antibodies can be tested using standard immunological assays, such as ELISA or Western blot, according to methods well known in the art (see, for example, Harlow and Lane, 1988, Antibodies: A Laboratory Manual, Cold Spring Harbor Laboratory Press, Cold Spring Harbor, N.Y.). In some situations, the drawn blood may be treated by apheresis techniques in which the blood is initially separated into components, i.e. white blood cells, and the components further processed for special purposes such as removing toxic or unwanted substances from the blood.

Isolating Immunocompetent or Non-Naive Lymphocytes/Antibodies

A column or support is prepared to extract or isolate the non-naive lymphocytes that produce non-naive antibodies from the blood or suitable blood component. A portion of the antigen sample used to immunize the animal is immobilized on a support or solid surface, i.e. magnetic beads, for subsequent panning or affinity chromatography. Antigens are immobilized by covalently linking them to the support or solid surface using selected conjugation methods and organic reagents that can conjugate certain chemical moieties, for example, the carboxy (—COOH) moieties, amino (—NH₂) moieties, periodate (glycoprotein) moieties and/or cysteine (mercaptol or thiol) moieties of the antigen proteins to the support or solid surface. For example, magnetic beads can be prepared in which the antigen proteins are covalently linked to the beads. A buffered blood or blood component or extract is added to the beads and mixed. The vast majority of B-cells in the extract can be washed off or away from the beads, since these cells fail to bind or bind weakly with the antigen protein. However, some of the B-cells will not wash off, and instead, bind to the beads. These binding B-cells express an antibody protein on their cell surface corresponding to the protein antigen of interest. B-cells bound to the beads can be isolated or removed by any of several eluting procedures and recovered as they elute or wash off the beads. Thus, the lymphocytes (e.g. plasma cells) recovered from the beads will be limited substantially to those lymphocytes producing immunocompetent antibodies to the source of antigen(s). Compared with naive B-cells, these specific B-cells are immunocompetent (i.e. non-naive) having undergone class switching and are able to produce useful, high affinity antibodies.

Preparing the Phage Display Library

A phage display library can be produced by constructing a cDNA expression library from a pool of RNA collected or recovered from animal lymphocytes (i.e. plasma cells) of interest. The molecule RNA codes for antibody proteins or fragments thereof generated by the lymphocyte cells. The above correlation between serum levels of antigen-specific antibodies, the number of lymphocytes producing those antigen-specific antibodies, and the amount of total mRNA encoding the antigen-specific antibodies provides a means for isolating a pool of mRNA that is enriched for the mRNA encoding antigen-specific antibodies of interest. The total RNA is then extracted from these lymphocytes. Methods for RNA isolation from animal cells can be found, for example, in Sambrook et al., 1989, Molecular Cloning: A Laboratory Manual, 2nd Ed., Cold Spring Harbor Laboratory Press, Cold Spring Harbor, N.Y. For example, the recovered B-cells of the immunized animal can be treated with a lysing reagent, such as dilute alkali under isotonic conditions, that ruptures the B-cell membrane and allows dissolution of the cell and exposure of the RNA. RNA is then isolated and subjected to a process known as reverse transcription to produce a cDNA library of the light and heavy protein chains that compose or make up an individual antibody. In reverse transcription, cDNA is synthesized and amplified from its RNA template using the enzyme reverse transcriptase and suitable oligonucleotide primers in the polymerase chain reaction (PCR). The 3′ primers used to amplify heavy chain or light chain-encoding cDNAs can be based upon the known nucleotide sequences common to heavy chain or light chain antibodies of a specific antibody subclass. The 5′ Primers can be consensus sequences based upon examination of a large number of variable sequences in the database. In this manner, DNA encoding all antibodies of a specific antibody class or subclass are amplified regardless of antigen-specificity of the antibodies coded by the amplified DNA. The entire gene encoding the heavy chain or the light chain can be amplified. Alternatively, only a portion of the heavy or light chain encoding gene may be amplified, with the proviso that the product of PCR amplification codes a heavy or light chain gene product that can associate with its corresponding heavy or light chain and function in antigen binding.

The synthesized cDNA is inserted into an appropriate phage display vector, such as the single-stranded DNA (ssDNA) filamentous bacteriophage M13, fl, fd, or other equivalent filamentous phage. After insertion of the cDNA, the phage display vector contains the genes encoding the heavy and light protein chains of the antibody or antibody fragment thereof. The cDNA may also be subjected to site directed mutagenesis prior to insertion into the display vector. Specifically, codons are removed or replaced with codons expressing different amino acids in order to create a larger library (i.e., a library of many variants) which is then expressed on the surface of the phage. Thereafter, the phage are brought into contact with a culture of phage-susceptible bacteria (such as a strain of E. coli) for infection by the phage. After infection, the phage replicate within the bacterial cell, usually making tens or even hundreds of copies of itself. The newly replicated phage will express or “display” antibody proteins or fragments thereof as fusion proteins on its surface or protein coat. These antibody proteins or fragments thereof are the same as antibodies produced by non-naive B-cells recovered from the immunized animal. In addition, the gene encoding the antibody or antibody fragment is inside the phage. After phage replication, the bacterial cells are lysed, either naturally by the phages or by chemical treatment, and the lysate is isolated from the bacterial cell debris. The phage lysate contains a collection or “library” of phages containing antibody genes (i.e. DNA) which code for immunocompetent or non-naive antibodies or fragments thereof produced by the immunization of a animal for a particular source of antigens. The library may also be described by saying that the lysate contains the filamentous phage expressing on its surface the cloned heavy and light antibody chains produced by the immunized animal. In theory, the heavy and light chains are present on the phage surface as Fab antibody fragments, with the heavy chain of the Fab being anchored to the phage surface via the filamentous phage membrane anchor portion of the fusion polypeptide. Additional presentation of the phage display can be done through use of a single chain fragment known as Fragment V (scFv), which is the smallest binding fragment of an antibody. The scFv is comprised of a single variable heavy chain linked to a single variable light chain. The scFv is a portion or about a quarter of a functional fragment antigen binding (Fab). Fab is comprised of an antibody lacking its complement binding and Fc binding domains. The light chain is associated with the heavy chain so as to form an antigen binding site. In either description, this library is referred to as a immunocompetent phage display library. In one embodiment, the immunocompetent phage display library may be prepared from scFv fragments created from specific B-cells. The phage display technology of the present invention provides a means for expressing a diverse population of antigen proteins specific to a source of multiple non-purified antigens resulting from a particular treatment or experimental set of conditions. Such immunocompetent phage display libraries are useful for proteomics, for mode of action studies and for development of assays.

Screening or Selecting Antigen Specific Antibodies from the Phage Display Library

Phage expressing an antibody or fragment thereof that specifically binds an antigen can be isolated using any of a variety of protocols for identification and isolation of monoclonal and/or polyclonal antibodies. Such methods include, immunoaffinity purification (e.g., binding of the phage to a column having bound antigen) and antibody panning methods (e.g., repeated rounds of phage binding to antigen bound to a membrane or solid support for selection of phage of high binding affinity to the antigen). For example, screening of a phage display library developed from the recovered B-cells or antibodies may involve panning the library using target molecules (i.e. antigen proteins separated and/or isolated using 2-D gels) on a PVDF membrane. Phages that bind the target molecule can be recovered, individual phage can be cloned and the peptide expressed by a cloned phage can be determined. In one procedure, phages are labeled with a fluorescent or radioactive tag to identify the phages. The PVDF membrane or excised proteins are panned with the entire library of labeled phages to hybridize antigenic proteins on the membrane with phages having complementary antibody proteins in the phage coat. The membrane can be the stained and the stained spots excised. Alternatively, the membrane can be scanned for labeled phages that have hybridized with antigenic proteins. The labeled or stained spots containing a complex of phage/antigen can be eluted or excised from the membrane. These eluted or excised spots provide a highly useful library of antibodies that can be further characterized in a variety of techniques, including proteomics (i.e. the protein complement of the genome) on a chip (protein expression profiling or PEP). Also, once the animal antibody genes have been isolated and cloned, this approach could be repeated very rapidly for any tissue of interest.

After identification and isolation of phages expressing antibodies to the multiple antigens, the phage can be used to infect a bacterial culture, and single phage isolates identified. Each separate phage isolate can be again screened using one or more of the methods described above. In order to further confirm the affinity of the phage for the antigen, and/or to determine the relative affinities of the phage for the antigen, the DNA encoding the antibodies or fragments thereof can be isolated from the phage, and the nucleotide sequence of the heavy and light chains contained in the vector determined using methods, as described for example, in Sambrook et al., supra.

In addition, methods are available for screening libraries of molecules to identify those of interest. For example, phage peptide display libraries can be used to express large numbers of peptides that can be screened in vitro with a particular target molecule or a cell of interest in order to identify peptides that specifically bind the target molecule or the cell. Screening of such phage display libraries has been used, for example, to identify ligands that specifically bind various antibodies and cell surface receptors.

The identification of a peptide from a phage display library using an in vitro panning or immunoaffinity purification method can represent a starting point for determining whether the identified peptide can be useful for an in vivo procedure.

In a preferred embodiment, a human phage display library is developed for a variety of tissues, organs, and fluids. Such development involves the capture of human B-cells from whole blood of donors. Donors can be pre-selected based upon their indication, cell surface antigens, Ab expression, etc. Such human phage libraries can used for Ab therapeutics, drug discovery, and diagnostics. It could also mimic the tremendous success in the dendritic cell world. Dendritic cells have recently been discovered to be involved in protein/peptide processing and subsequent presentation to B-cells. They have the ability to recognize cancer cells as foreign even though the normal APCs do not. Researchers and physicians have demonstrated that dendritic cells removed from a patients blood and pulsed with known cancer antigens (peptides) can be re-infused back into the patient whereby a previously non-existent immune reaction against the cancer cells begins. In some instances, with well characterized cancers, complete remission has resulted in up to 80% of the trial subjects. In any case, with such a human library the uses are virtually without limit.

Separating the Antigen Proteins and their Characterization

The proteins in the antigenic source may be characterized by protein/peptide fingerprinting. In peptide fingerprinting, a single protein may be characterized from its component peptides, which can be easier to characterize and sequence than the whole protein. Initially, proteins in the antigenic source are separated from one another by using a technique known as two-dimensional (2-D) gel electrophoresis, a multistep electrophoretic process for resolving complex mixtures of proteins. The proteins are separated in a slab of gel on the basis of their apparent molecular weights and electrical charge (i.e. isoelectric point). The proteins in a sample are first broken down to denatured polypeptide subunits by dissolving in either Urea or SDS buffer. The solution is generally held in a porous support medium such as filter paper, cellulose acetate (rayon) or a gel made of starch, agarose, polyacrylamide or mixtures of agarose and polyacrylamide. The polypeptides are exposed to an electric field, causing the proteins in the antigenic source to separate individually along the length of the gel plate or 1-Dimension (1-D), based upon their mobility in an electric field, using for example, SDS PAGE, whereby the proteins are exposed to the anionic detergent sodium dodecyl sulfate (SDS) and polyacrylamide gel electrophoresis (PAGE). When the SDS binds to proteins, it breaks all noncovalent interactions so that the protein molecules assume a random coil confiuguration, provided no disulfide bonds exist (the disulfide bonds can be broken by treatment with mercaptoethanol). Frictional resistance produced by the support causes size, rather than charge alone, to become a major determinant of separation. An electric field in then applied at a right angle to the length of the 1-D pattern to produce a two-Dimension (2-D) pattern of polypeptide or protein spots which are characteristic of a mixture of proteins as a fingerprint. The distance moved per unit time by a random coil follows a mathematical formula involving the molecular weight of the molecule, from which the molecular weight can be calculated. After the tissue extracts have been separated into individual proteins on the 2-D gel, the proteins can be transferred to membrane material such as polyvinylidene difluoride (PVDF), nylon or nitrocellulose. PVDF has less binding capacity but is physically stronger than nitrocellulose. PVDF also has less non-specific binding than nylon and is also very amenable to MALDI-TOF protocols. The PVDF membrane can be stained with a reagent that will help visualize or detect the protein and/or peptides, such a Ponceau S, Coomassic or Ruby Red, i.e. dyes used to stain proteins. Additionally, the proteins on the membrane should be partially digested using protease enzymes that will cut or break up the individual proteins into smaller peptides that are more amenable to analysis by mass spectrometry. For example, trypsin is commonly incubated in a membrane, such as PVDF, to give a trypsinized memberane, whereby the resultant peptides maintain their physical location.

Analyzing Proteins and/or Peptides Thereof

Once the protein and/or peptides spots on the 2-D gels and/or PVDF membranes are detectable, they can be excised and isolated for further characterization. A major tool for their characterization is the mass spectrometer (MS). A mass spectrometer measures the exact molecular mass of a molecule by measuring its flight path through a set of magnetic and electric fields. More specifically, the MS measures the mass/charge ratio. If one already has a database of known protein sequences, the identity of an unknown protein can be determined by its atomic weight, since mass spectrometry can accurately measure the atomic weight. Tandem MS (MS/MS) coupled with various ion sources such as ESI (electrospray ionization) or MALDI (matrix assisted laser desorption ionization) can effectively determine both mass and peptide sequence, thus providing valuable information in the field of proteomics. The ions from these ion sources are directed into a mass analyzer, which could be either a triple-quadrupole, an ion trap, a Fourier-transform ion cyclotron resonance (FT-ICR) or a hybrid quadruple Time-of-Flight (TOF).

Additional determinations of protein and/or peptide sequence for the antigen protein can be supplemented with searches of homologous protein sequence in publicly available databases using programs such as BLAST and FASTA.

Preparing Monoclonal Antibodies

In a different approach, antibodies can be made which are said to be monoclonal, i.e. the product of a single hybrid clone of B-lymphocytes and typically a myeloma cell line (hybridoma). Monoclonal antibodies are secreted by hybridoma cells, which can be prepared according the procedures described in Basic Methods in Antibody Production and Characterization, eds. (G. C. Howard and D. R. Bethell), Chapter 6, Sandy J. Stewart, Monoclonal Antibody Production, CRC Press LLC, Boca Roca Fla., 2000, pp. 51-67. In one alternative, large animals such as those described before, are immunized with the tissue of interest (i.e. the antigenic source) to produce the initial antibodies using the immunization procedures described above. After achieving a high antigenic titer, B-cell containing tissue such as the spleen, lymph nodes and thymus are surgically removed and homogenized to release as many B-cells as possible into the solution while attempting to minimize cell death. Monoclonal antibody-secreting hybridoma cells are prepared by fusing a single B-cell with a myeloma cell using polyethylene glycol (PEG), culturing the fused in a suitable animal cell culture media, screening the cultured cells and cloning selected cultured hybridoma cells. The proteins in the antigen preparation, formulation or extract can be separated via 2-D gel electrophoresis and the resultant monoclonal antibodies (mAbs) are screened against the separated antigenic proteins in the 2-D gel for a quick primary screen. The antigenic proteins can be subsequently screened using Western blotting, a procedure in which proteins or peptides are transferred from an electrophoresis gel to a rigid membrane support, such as PVDF. The antigenic proteins are identified through spots formed by the binding of a labeled monoclonal antibody to antigenic protein or peptide molecules. The spots can be excised and characterized via MS/MS and/or MALDI-TOF analysis, described above.

### EXAMPLE

The following example is put forth so as to provide those of ordinary skill in the art with a complete disclosure and description of how to make and use the present invention, and is not intended to limit the scope of the invention. Efforts have been made to ensure accuracy with respect to numbers used (e.g. amounts, temperature, etc.) but some experimental errors and deviations should be accounted for.

Preparation of naive phage display library. Use normal non-immune chicken B-cells to produce phage library. Isolate mRNA from naïve B cells and prepare cDNA. Design primers specific for all major heavy and light chicken immunoglobulin chains. PCR the variable heavy (VH) and light (VL) regions from above cDNA for each isotype. Join VH and VL cassettes with PCR “SOEing” (Splicing of Overlapping Ends) such that the two are covalently linked into one chimeric gene (single chain Fv); linkage is specific for each isotype. Repeat process such that each isotype is represented in the resulting pool. Clone scFv genes into phage vectors, transform E. coli. Rescue phage to create a naive phage display library, prepare stocks.

Preparation of immunocompetent phage display library. Perform crude protein extractions on human plasma and analyze via 2-D gels to identify best overall efficiency. Use chromatography to remove globulin factor prior to treatment with 2-D gels. Identify as many of the proteins using Maldi-MS/MS. Immunize several chickens with this same extract. Bleed the chickens and isolate immunized high titered chicken B-cells using ficoll-hypaque gradient followed by separation of immunocompetent B-cells of interest with magnetic beads conjugated with above extract using N-terminal, C-terminal, and glutaraldehyde methods. With these immunocompetent B-cells, isolate mRNA and prepare cDNA. Rescue phage to create a specific, immunocompetent phage display library, prepare stocks. Produce immunocompetent phage display library using isolated DNA from specific B-cells. Screen library on Western blots of above with plasma extract using conventional methods. Stain PVDF membrane with Coomassic blue prior antibody (Ab) hybridization. Recover phage DNA from Coomasic stained or amido block stained PVDF by simply scraping/excising spot. Reconstitute phage particles by transforming E. coli with isolated DNA. Resulting plaques are representative of phage that bound the original PVDF associated protein DNA sequence of recovered clone is compiled and translated into putative protein. Test identified antibodies against Westerns and ELISA using same plasma extract. Microarray antibodies and test against plasma extract.

What is claimed is:

1. A method for preparing an immunocompetent phage display library for a tissue of interest comprising:
a) injecting into an animal,a source of antigen(s) from an organism of interest;
b) allowing said animal to develop specific B-cells expressing predominately (primarily) immunoglobin IgG antibody against said antigen(s);
c) withdrawing a blood sample from said animal containing said B-cells expressing immunoglobin on their cell surface;
d) immobilizing the source of antigen(s) on a support or solid surface;
e) contacting said blood or blood component with said support or solid surface to capture said specific B-cells expressing IgG directed against said antigen(s) of interest from said blood or blood component; and
f) recovering said specific B-cells from said support or solid surface; and
g) preparing an immunocompetent phage display library from said recovered specific B-cells.

2. The method of claim 1 wherein said animal is a chicken or sheep.

3. The method of claim 1 wherein for step g) the immunocompetent phage display library is prepared from scFv fragments created from said specific B-cells.

4. A method for preparing a immunocompetent phage display library, comprising:
a) mounting an immune response in an animal, with an a source of multiple antigens from a tissue of interest to generate specific B-cells that have undergone in vivo somatic hypermutation,
b) recovering specific B-cells from the blood or blood component of the immunized animal, and
c) preparing an immunocompetent phage display library that displays antibody proteins or fragments thereof, from the recovered, specific B-cells.

5. The method of claim 4 wherein for step c) the immunocompetent phage display library is prepared from scFv fragments created from said specific B-cells.

6. An immunocompetent or non-naive phage display library per se prepared by the method of claim 1.

7. An immunocompetent or non-naive phage display library per se prepared by the method of claim 4.

8. A method for screening for phages within an immunocompetent phage display library comprising:
a) mounting an immune response in an animal with an a source of antigen(s) from an organism of interest to generate specific B-cells that have undergone in vivo somatic hypermutation,
b) isolating said specific B-cells from the blood or blood component of the immunized animal,
c) preparing a phage display library that displays antibody proteins or fragments thereof from said specific B-cells,
d) separating said source of antigen(s) into individual proteins or peptides thereof, and
e) screening or selecting for phages within said immunocompetent phage display library that bind with said separated antigen proteins or peptides.

9. The method of claim 8 wherein for step c) the phage display library is prepared from scFv fragments created from said specific B-cells.

10. A method for characterizing either the phages within an immunocompetent phage display library and/or its antigen source, comprising:
a) mounting an immune response in an animal with an a source of antigen(s) from an organism of interest to generate specific B-cells that have undergone in vivo somatic hypermutation;
b) recovering said specific B-cells from the blood or blood component of the immunized animal;
c) preparing a phage display library that displays antibody proteins or fragments thereof, from said specific B-cells,
d) separating said source of antigen(s) into individual proteins or peptides thereof,
e) screening or selecting for phages within said immunocompetent phage display library that bind with said separated antigen proteins or peptides, and
f) characterizing:
i) the protein sequence and/or molecular weight of the separated antigen proteins binding to the phage and/or
ii) the nucleic acid sequence within the binding phage that codes for the antibody protein or fragment thereof.

11. The method of claim 10 wherein for step c) the phage display library is prepared from scFv fragments created from said specific B-cells.

12. A method for preparing a library of antibodies and/or fragments thereof, comprising:
a) injecting into an animal a source of antigen(s) from an organism of interest;
b) allowing said animal to develop specific B-cells expressing predominately immunoglobin IgG antibodies;
c) withdrawing a blood sample from said animal containing said specific B-cells;
d) immobilizing the source of antigen(s) to a support or solid surface;
e) contacting said blood sample or component thereof with said support or solid surface to remove said specific B-cells from said blood or blood component;
f) isolating RNA from said specific B-cells;
g) reverse transcribing said RNA to produce cDNA;
h) inserting said cDNA into a phage display vector that expresses antibodies on the phage surface,
i) isolating phage which bind to said source of antigen(s), and
j) obtaining a library of antibodies or fragments thereof from said phage.

13. A method for preparing a library of antibodies and/or fragments thereof, comprising:
a) mounting an immune response in an animal with an a source of antigen(s) from a tissue of interest to generate specific B-cells that have undergone in vivo somatic hypermutation,
b) isolating specific B-cells from the blood or blood component of the immunized animal,
c) preparing an immunocompetent phage display library that displays antibody proteins or fragments thereof from said specific B-cells,
d) separating said source of antigen(s) into individual proteins or peptides thereof,
e) screening or selecting for phages within said immunocompetent phage display library that bind with said separated antigen proteins or peptides, and
f) obtaining antibodies or fragments thereof from said binding phage.

14. A library of antibodies per se prepared by the method of claim 12.

15. A library of antibodies per se prepared by the methods of claim 13.
